(19) United States
(12) Patent Application Publication
(10) Pub. No.: US 2008/0133014 A1
(43) Pub. Date: Jun. 5, 2008

(54) ARTIFICIAL INTERVERTEBRAL DISC ASSEMBLY AND METHOD FOR ASSEMBLY WITHIN SPINE

(75) Inventors: Nicholas V. Gately, Lambertville, NJ (US); Jeffry Howard Nelson, Fairfax, VA (US)

Correspondence Address: NIXON & VANDERHYE, PC, 901 NORTH GLEBE ROAD, 11TH FLOOR, ARLINGTON, VA, 22203, US

(73) Assignee: G&L Consulting, LLC, New York, NY (US)

(21) Appl. No.: 11/567,012

(22) Filed: Dec. 5, 2006

(51) Int. Cl. A61F 2/44

(52) U.S. Cl. 623/17.16; 623/17.15

(57) ABSTRACT

A method to insert an artificial disc in the spine of a patient including: sequentially inserting at least two components of an artificial disk in an vertebral interbody space; assembling the components within the vertebral interbody space to form an artificial disc, and using the assembled artificial disc as a disc in the vertebral interbody space. The interbody space is between two adjacent vertebra. The components of the artificial disc are sequentially surgically inserted posteriorly into the patient or sequentially inserted with a lateral surgical approach, and are surgically inserted without resection of facet joints in the spine.

This application claims the benefit of U.S. Provisional Application Ser. Nos. 60/743,013 filed Dec. 5, 2005, and 60/743,065 filed Dec. 21, 2005, the entirety of both which applications are incorporated herein by reference.

### BACKGROUND OF THE INVENTION

The present invention generally relates to spinal implants inserted in the spine of a patient during surgical procedures and to method for surgically inserting the implants.

A spinal implant may be used to stabilize a portion of a spine. The implant may promote bone growth between adjacent vertebra that fuses the vertebra together. The implant may include a spherical protrusion, a threaded pin and an angled surface to facilitate remote adjustment of the implant position using an insertion instrument.

An intervertebral disc may degenerate. Degeneration may be caused by trauma, disease, and/or aging. An intervertebral disc that becomes degenerated may have to be partially or fully removed from a spinal column. Partial or full removal of an intervertebral disc may destabilize the spinal column. Destabilization of a spinal column may result in alteration of a natural separation distance between adjacent vertebra. Maintaining the natural separation between vertebra may prevent pressure from being applied to nerves that pass between vertebral bodies. Excessive pressure applied to the nerves may cause pain and nerve damage. Artificial discs maintain spacing and articulation between vertebral bodies normally allowed by the elastic properties of the natural disc, which directly connects two opposing vertebral bodies. Various artificial discs are described by Stefee et al. in U.S. Pat. No. 5,071,437, and Gill et al. in U.S. Pat. No. 6,113,637.

During a spinal fixation procedure, a spinal implant may be inserted in a space created by the removal or partial removal of an intervertebral disc between adjacent vertebra. The spinal implant may maintain the height of the spine and restore stability to the spine. Bone growth may fuse the implant to adjacent vertebra. Spine fusion helps eliminate pain, but limits the range of spinal motion for patients. It is thought that spine fusion creates increased degeneration in adjacent non-fused segments, commonly known as “adjacent segment disease.”

Traditional artificial disc surgery treatment requires removal of the natural human disc and the insertion of two plates through the abdomen, i.e. the anterior side of the patient. Anterior insertion of the disc necessitates surgical dissection of the two major blood vessels, the vena cava and the aorta. Rupture of these blood vessels are a life threatening complication of an anterior approach to the lumbar spine, as well as nerve damage resulting in retrograde ejaculation in males. Because of the risks, such surgery requires the attendance of both a general surgeon as well as an orthopedic surgeon.

As a result, there is a need for an artificial disk which may be inserted unilaterally (or bilaterally) from the posterior side of the patient.

### BRIEF DESCRIPTION OF THE INVENTION

An artificial disk and surgical insert method have been developed in which the disk components are inserted posteriorly and assembled in situ, e.g., within the vertebral interbody space. The components of the disk are small enough that combined with a lateral surgical approach, resection of the facet joints and manipulation of the spinal cord may be completely avoided. Other surgical techniques may be employed for the insertion of the disc components and assembling the components in situ. Assembling in situ allows for a fully functioning artificial disk to be inserted posteriorly.

In another embodiment, a spinal implant may be inserted during a spinal fixation procedure using an anterior, lateral, posterior, or transverse spinal approach. A discectomy may be performed to remove or partially remove a defective or damaged intervertebral disc. The discectomy may create a space for a spinal implant. The amount of removed disc material may correspond to the size and type of spinal implant to be inserted.

A method to insert an artificial disc in the spine of a patient has been developed that comprises: sequentially inserting at least two components of an artificial disk in an vertebral interbody space; assembling the components within the vertebral interbody space to form an artificial disc, and using the assembled artificial disc as a disc in the vertebral interbody space. The interbody space is between two adjacent vertebra. The components of the artificial disc are sequentially surgically inserted posteriorly into the patient or sequentially inserted with a lateral surgical approach, and are surgically inserted without resection of facet joints in the spine.

A method has been developed for forming an artificial spine disc assembly including: an upper spine support plate and lower spine support plate, wherein each plate has a generally planer surface adapted to face and engage a vertebra and a first coupling; an upper disc beam and a lower disc beam, each beam comprising one of a pair of opposing joint surfaces and second coupling adapted to connecting to one of the first coupling to a respective one of the support plates, the method comprising: surgically and individually inserting the disc beams into vertebral interbody space in a spine of a patient; assembling the upper disc beam and the lower disc beam by mating the opposing joint surfaces in the vertebral interbody space, and surgically and individually inserting the support plates into vertebral interbody space in a spine of a patient; attaching the coupling of each support plate to a corresponding one of the disc beams in the vertebral interbody space. Wherein the upper support plate is a pair of upper support plates and the lower support plate is a pair of lower support plates, such that attaching of the support plates includes attaching each of pair of upper support plates to opposite beams on the upper disc beam and attaching each of a pair of lower support plates to opposite beams on the lower disc beam. The method further comprises positioning the upper support plate opposite to a lower surface of an upper vertebra and positioning the lower support plate opposite to an upper surface of a lower vertebra.

An artificial spine disc assembly has been developed comprising: a first disc beam having a center block with a rounded joint surface and a pair of opposite beams extending outward from the center block; a second disc beam having a center block with a second joint surface and a pair of opposite beams extending outward from the center block, wherein the second joint surface seats into the first joint surface when the second disc beam is mounted on the first disc beam; a first pair of spine support plates, wherein each plate has a generally planer surface adapted to face and engage a vertebra and a coupling to engage one of the opposite beams of the first disc beam, and a second pair of spine support plates, wherein each plate has a generally planer surface adapted to face and engage a vertebra and a coupling to engage one of the opposite beams of the first disc beam. In the artificial spine disc assembly, the first joint surface is a concave surface in an upper face of the center block of the second disc beam, and the second joint surface is a convex surface on a lower face of the center block of the first disc beam. The concave surface is a rounded well in the upper face and the convex surface is a semi-hemispherical surface in the lower face. The artificial spine disc assembly is packaged with the disc beams and spine support plates disassembled in a sterile package to be shipped to a medical service provider.

### BRIEF DESCRIPTION OF THE DRAWINGS

FIG. 1 is a perspective exploded view of an artificial intervertebral disc assembly.

FIG. 2 is a cross-sectional view of the disc assembly shown in FIG. 1.

FIG. 3 is a perspective view of the disc assembly shown in FIG. 1, without the pads.

FIG. 4 is an end view of the disc assembly shown in FIG. 3.

FIG. 5 is a cross-sectional view of the disc assembly shown in FIG. 3 and taken along line 5-5 in FIG. 4.

FIG. 6 is a top, front and side perspective view of a component of the disc assembly shown in FIG. 3.

FIGS. 7 to 14 illustrate a surgical method for inserting the components of the disc assembly between adjacent vertebra, and assembling the components in the space between the vertebra to form an artificial intervertebral disc.

FIG. 15 is a sterile bag with the separate components of the artificial disc assembly so that they can be removed during surgery and the components are sequentially inserted into the spine of a patient.

### DETAILED DESCRIPTION OF THE INVENTION

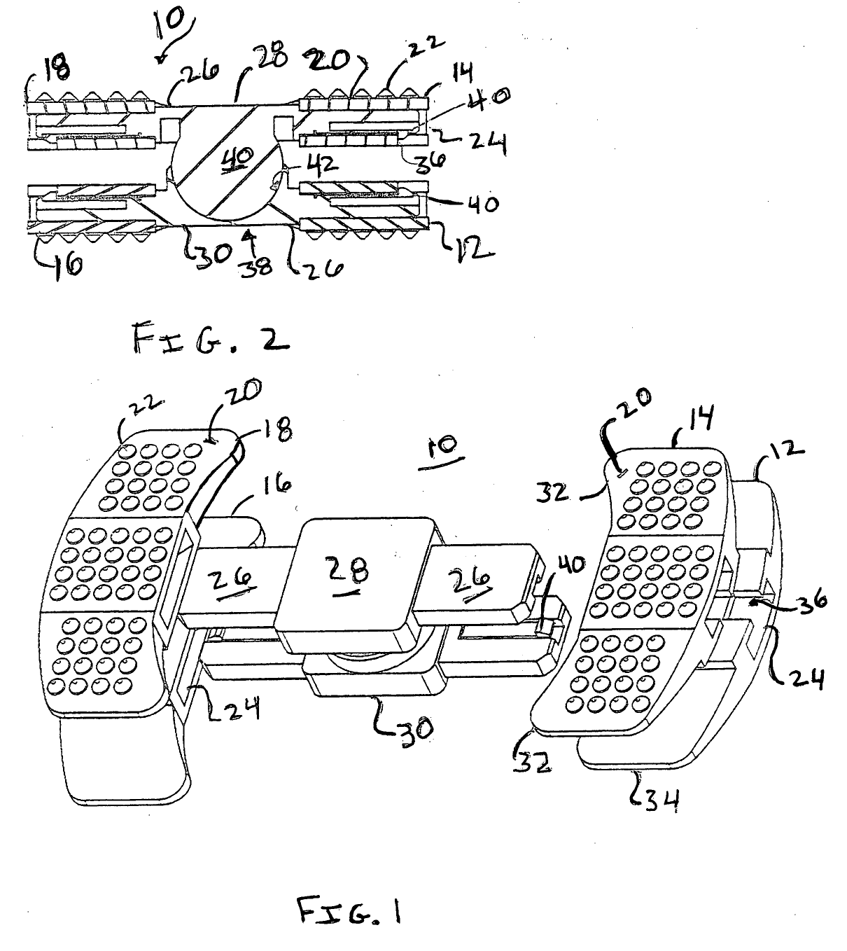

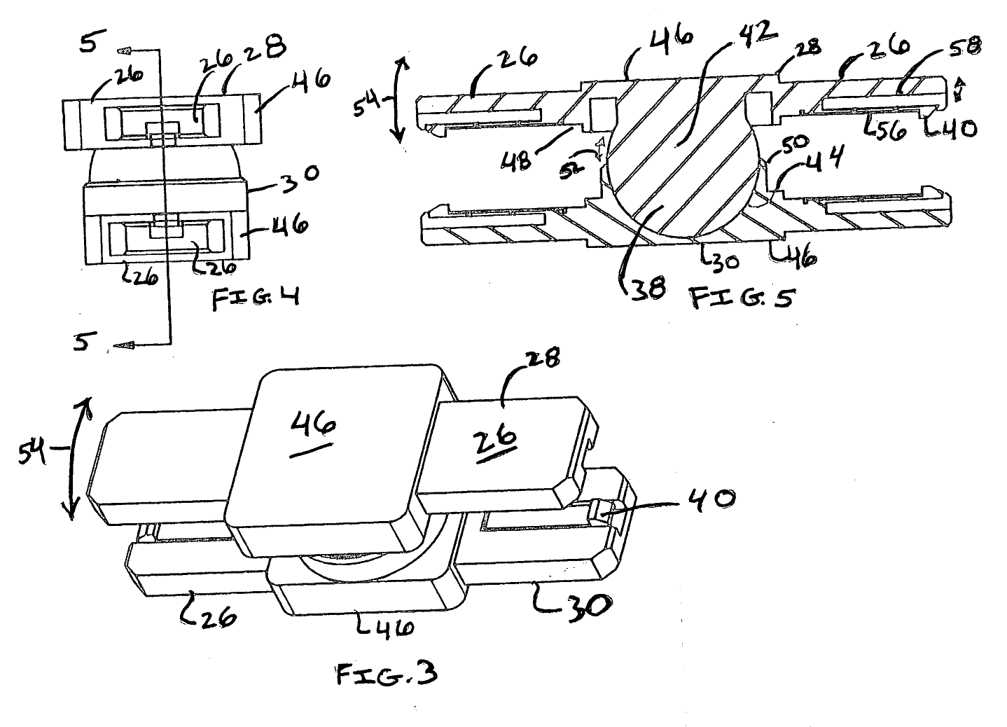

FIG. 1 is an exploded view of an artificial intervertebral disc assembly 10. FIG. 2 is a cross-sectional view of the disc assembly. The assembly fits between opposing vertebra and functions as an artificial disc between the vertebra. The assembly provides pivoting movement and deformation to approximate the movement and deformation of a natural disc between vertebra.

The assembly 10 includes four spine support plates, 12, 14, 16 and 18. Each plate has a generally planer surface 20 that faces and engages an end of vertebra. The upper plates 14, 18 engage the lower end surface of an upper vertebra. The upper plates are positioned towards opposite sides of the upper vertebra end surface. The lower plates 12, 16 engage the upper end surface of a lower vertebra. The lower plates are positioned towards opposite sides of the lower vertebra end surface. The surface 20 of the plates 12, 14, 16 and 18 may include bumps 22, rough textured surface features or other surface treatments to promote adhesion between the surface and the end surface of the vertebra. The surface 20 of the plates may have a geometry that generally conforms to the end surface of the vertebra and provides a good amount of surface area to spread out loads applied between the vertebra.

The plates have a center slot 24 to receive a cantilever arms 26 from a disc beam 28, 30. The center slot may be in a relatively thick portion of the plates to ensure adequate structural support in the plates. The plates may have wings 32 extending from the center slot and become gradually thinner as they extend to an outer plate edge 34. The center slot may include an end lip 36 that may be a rectangular cutout in the end of the slot. The end lip receives a cantilever latch 40 on the arms 26 of the disc beam. The end lip and latch 40 snap together as the arm slides in through the slot 24. The secure engagement between the latch 40 and the lip 36 ensures that the plates do not slide off the arms after assembly of the artificial disc.

The beams may be integrally formed of a rigid biocompatible plastic material, e.g., Polyetheretherketone. The plates 12, 14, 16 and 18 may be formed of the same or other biocompatible plastic material.

When fully assembled, the artificial disc 10 replaces a spinal disc and fits between adjacent vertebra. The plate surfaces 20 of the upper and lower plates hold apart the vertebra. The disc beams hold the plates together and allow the plates limited movement, including pivoting and deformation. The deformation is provided by bending of the cantilevered arms 26 about the joint between the opposing disc beams 28, 30. Pivot movement is provided by the joint 38 formed by the insertion of a hemispherical ball joint 42 slidably fitting into a convex socket 44 cupped to receive the ball joint. The joint allows the ball to slide within the socket and thereby allows the upper plates to pivot with respect to the lower plates. The deformation and pivot movements of the artificial disc 10 replicate the natural movements between vertebra allowed by a healthy natural disc.

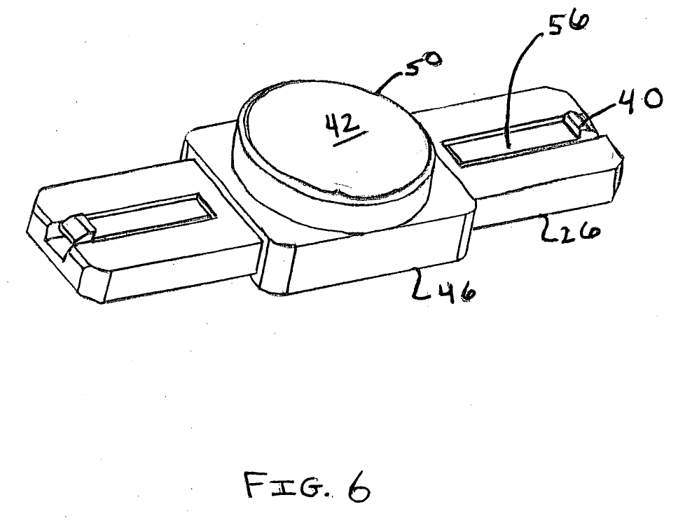

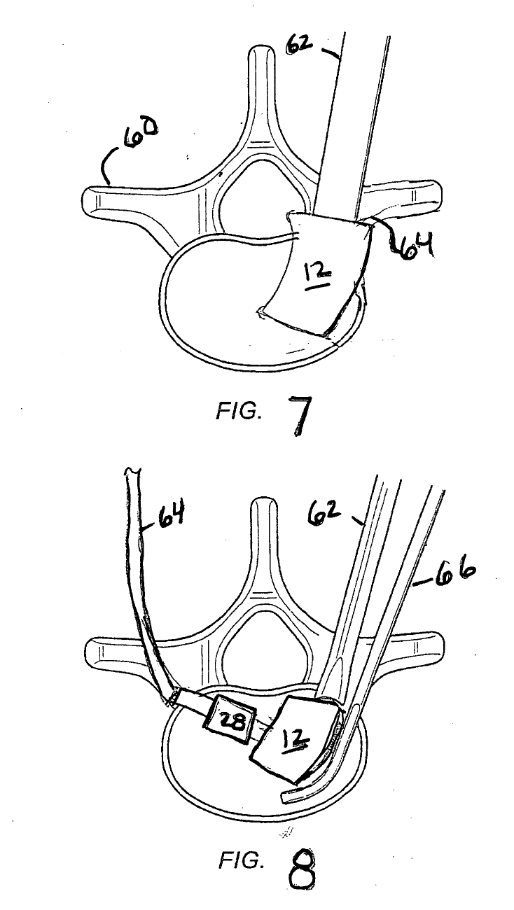

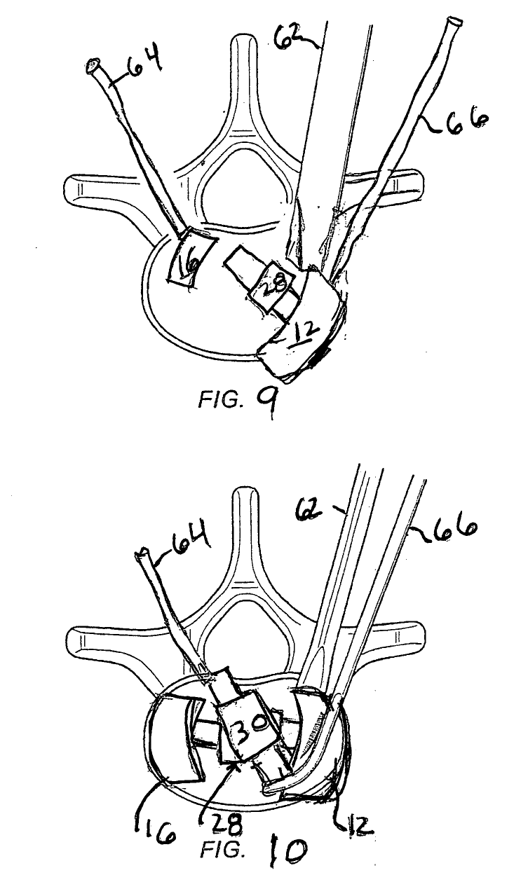

FIGS. 3, 4 and 5 are, respectively, a perspective view of a pair of disc beams 28, 30, an end view of the assembled disc beams and a cross-sectional side view of the assembled disc beams. The disc beams 28, 30 comprise a central rectangular base 46 and outwardly extending arms 26. The base 46 provides a support platform for the arms and for the joint 38 between the beams.

The arms 26 of each disc beam provide cantilever supports for the plates. The arms deform, e.g., bend, due to the pressure between the vertebra. The deformation provides a cushioning effect for the vertebra.

The base 46 for the disc beam 28 with the ball joint is relatively hollow with a peripheral walls extending around the four sides of the base. A ball joint 42 is seated within the hollowed out portion of the base 46. The base 46 for the disc beam 30 with the socket 44 is relatively solid but for a removed portion forming the socket. The base for the socket includes an annular ridge 50 having an inner curved surface that forms a portion of the socket. The materials for the ball and socket could be any combination of biocompatible ceramic, plastic (i.e. PEEK), stainless steel or wear resistant Cobalt-Chrome alloy.

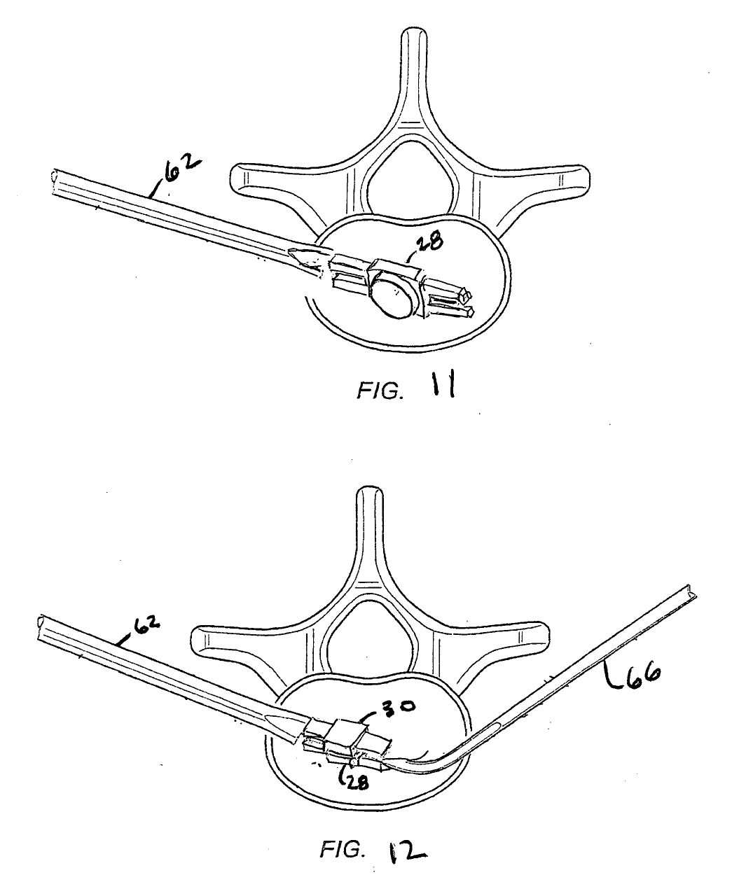

As shown in FIG. 6, the surface of the socket 44 conforms to the surface of the ball 42. A distal end of the ball seats in the socket and a substantial portion, e.g., one-half to two-thirds, of the ball extends out from the socket. The ball slidably engages the socket such that the upper disc beam 28 pivots with respect to the lower beam disk, as is indicated by arrow 52. The pivoting of the ball in the socket allows the upper plates 14, 18 (FIG. 1) to pivot and rotate with respect to the lower plates 12, 16. The pivoting movement between the upper and lower plates provides for movements similar those that occur between adjacent vertebra.

The latch 40 on each arm includes a beam 56 extending through a channel 58 in the arm. The cantilever beam 56 causes the latch 40 to snap into place against the lip 36 (FIG. 2) in the slot of a plate.

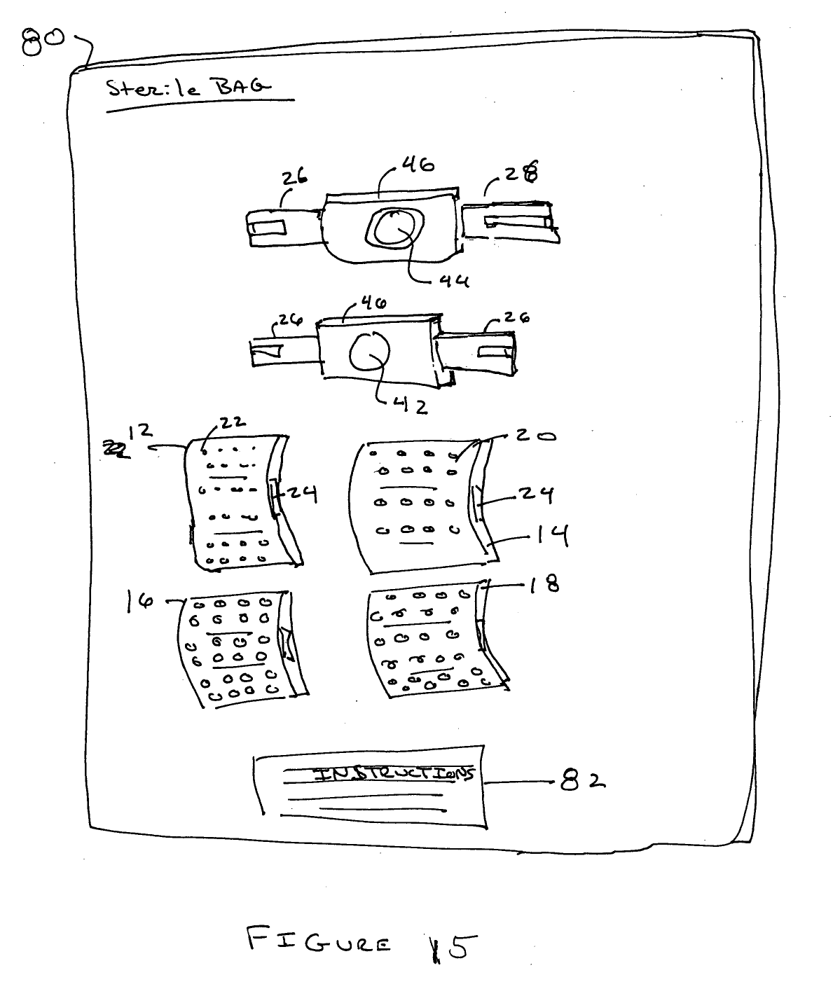

The artificial intervertebral disc assembly 10 may be assembled between the vertebra. The individual components of the disc assembly, e.g., disc beams and platforms, may be surgically inserted serially in between the vertebra. The components are assembled in place between adjacent vertebra. The components of the disc assembly are packaged in a sterile package (See FIG. 15), such as a vacuum sealed plastic bag 80 with the components mounted on a plastic board in the bag. Instructions 82 for assembly may be included in the bag or printed on the outside of the bag. The sealed package is shipped to a medical provider, such as a hospital and remains sealed and sterile until opened by a surgical team member in a sterile room, such as the operating room where the implants will be inserted into a patient.

The components of the disc assembly 10 may be inserted during a spinal fixation procedure using an anterior, lateral, posterior or transverse spinal approach. A posterior spinal approach through the back of a patient has an advantage of being relatively minimally invasive, especially as compared to an anterior spinal approach. The components of the disc assembly may be inserted and assembled using an endoscope.

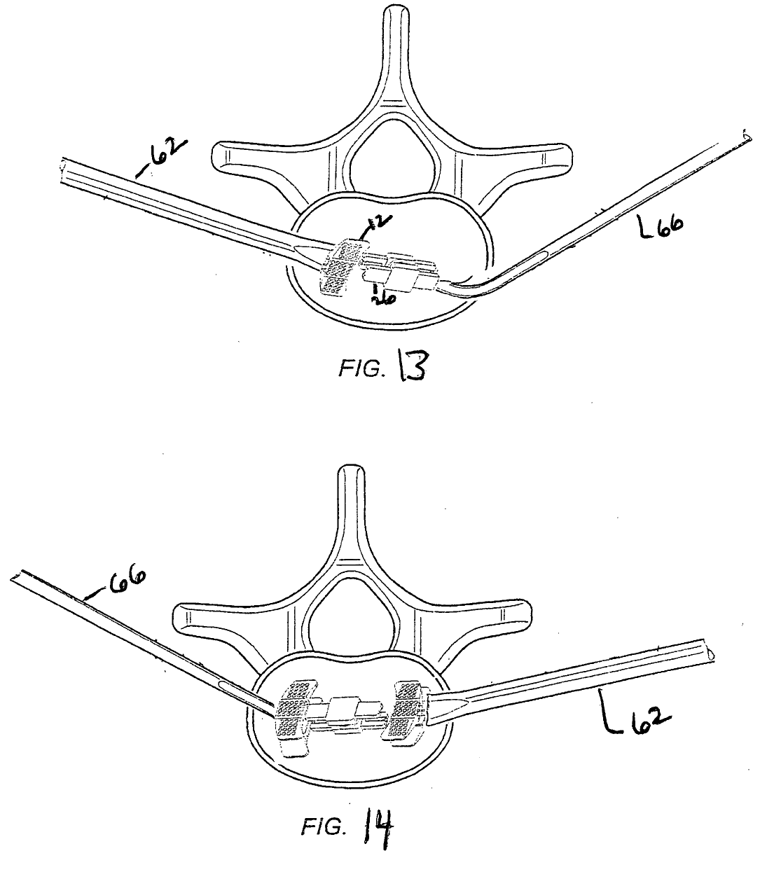

As is illustrated in FIGS. 7 to 14, a discectomy may be performed to remove a defective or damaged intervertebral disc, and to insert and assemble the components of the disc assembly 10. The discectomy creates a space for the disc assembly between the vertebra 60.

As shown in FIG. 7, an endoscope 62 is inserted in through an incision in the back of the patient and between adjacent vertebra 60. The endoscope includes a distal end comprising a releasable latch 64 for the plate. As shown in FIG. 7, the endoscope positions the plate between the vertebra and holds the plate to receive the arm of an disc beam 28.

As shown in FIG. 8, the disc beam 28 is inserted between the vertebra using an insertion rod 64. The disc beam may or may not be inserted on an opposite side of the vertebra than was the plate. A positioning rod 66 with a curved distal end may be used to assisting in relatively positioning the plate and disc beam, aligning the beam to receive the plate and sliding the arm of the disc beam into the plate. When the latch on the arm snaps into the plate 12, the insertion rod 64 is released from the beam and removed.

As shown in FIG. 9, the second plate 16 is inserted using the insertion rod 64, endoscope 62 and positioning rod 66. The second plate is slid onto the second arm of the disc beam 28. The second plate may be oriented such that the plates are aligned with opposite sides of the ends of the vertebra. The disc beam may have the ball 40 protruding upward.

As shown in FIG. 10, the assembly of the disc beam 28 and plates 12, 16 may be held in place by the endoscope 62. The insertion rod 64 may position the other disc bar 30 between the vertebra and onto the ball 40 of the first disc beam. Thereafter, the plate 14 may be inserted using the insertion rod and slid onto an arm of the disc beam. The positioning rod may rotate the disc beam 180 degrees so that the other arm of the disc beam can receive the other plate 18. Once the assembly of the artificial intervertebral disc is completed, the insertion rod and positioning rod align the assembly between the vertebra, and the endoscope releases the first plate 12.

An alternative surgical approach shown in FIGS. 11 to 14 is a modified transforaminal technique where access (see endoscope 62 and positioning rod 66) to the vertebral interbody space is sufficiently lateral that the facet joint is not resected, leaving the facet joints intact. Resection of a portion of the vertebral transverse process allows sufficient access to insert the components of the artificial intervertebral disc assembly and assemble them between the vertebra. This modified transforaminal procedure provides a more lateral surgical approach than is shown in FIGS. 7 to 10. This procedure would reduce the need to removal of the facets on both sides of the vertebra, as may be needed with the approach illustrated in FIGS. 7 to 10.

FIG. 11, shows an exemplary transforaminal technique in which an endoscope 62 (or other insertion tool) grips a disc beam 28 and inserts the beam through a lateral surgical incision in the patient and into an area between vertebra where the natural disc has been previously removed. The distal end of the endoscope has a releasable grip on an end of the disc beam, such as a latch, clamp, pair of hooks, screw or other attachment mechanism.

As shown in FIG. 12, the other disc beam 30 is moved into position between the vertebra and over the first disc beam 30. The ball and socket joint formed by the pair of disc beams is snapped into place when the disc beams are aligned. A positioning rod 66 may be used to hold the first disc beam 28 as the second disc beam 30 is moved into alignment. The positioning rod may be inserted through an incision on the opposite side of the patient's back from the incision used for the endoscope or the same incision may be used for both the endoscope and positioning rod.

With the disc beams in place and aligned, the first plate 12 is inserted through the incision by the endoscope 62 (or other insertion tool) such that its slot slides onto its respective arm 26 of one of the disc beams. A latch on the arm snaps into a groove in the plate to secure the plate to the arm. The positioning tool 66 may position the pair of disc beams such that the center slot in the plate is lined up with the arm of the disc beam. The second plate is inserted on its respective arm of the opposite disc beam.

As shown in FIG. 13, the partial assembly of the beams and two plates may be turned between the vertebra so that the endoscope can insert the remaining two plates. Alternatively and as is shown in FIG. 14, the endoscope 62 or other insertion device is removed from the first incision and inserted (with a plate) into the incision on the other side of the back of the patient. By switching the side at which the plates are inserted, the partial assembly of disc beams and plates need not be turned 180 degrees between the vertebra. With the endoscope on the other side of the back, the remaining two plates are inserted onto their respective arms of the disc beams in the same manner as used to slide the first two plates on their arms. The positioning rod 66 may be moved to the other side of the patient (which is the side initially used for the endoscope) to assist in snapping the remaining two plates into place.

While the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope of the appended claims.

We claim:

1. A method to insert an artificial disc in the spine of a patient, the method comprising:
sequentially inserting at least two components of an artificial disk in an vertebral interbody space;
assembling the components within the vertebral interbody space to form an artificial disc, and
using the assembled artificial disc as a disc in the vertebral interbody space.

2. A method as in claim 1 wherein the interbody space is between two adjacent vertebra.

3. A method as in claim 1 wherein the components are sequentially surgically inserted posteriorly into the patient.

4. A method as in claim 1 wherein the components are sequentially inserted with a lateral surgical approach.

5. A method as in claim 1 wherein the components are small and are surgically inserted without resection of facet joints in the spine.

6. A method for forming an artificial spine disc assembly including:
an upper spine support plate and lower spine support plate, wherein each plate has a generally planer surface adapted to face and engage a vertebra and a first coupling;
an upper disc beam and a lower disc beam, each beam comprising one of a pair of opposing joint surfaces and second coupling adapted to connecting to one of the first coupling to a respective one of the support plates,
the method comprising:
surgically and individually inserting the disc beams into vertebral interbody space in a spine of a patient;
assembling the upper disc beam and the lower disc beam by mating the opposing joint surfaces in the vertebral interbody space, and
surgically and individually inserting the support plates into vertebral interbody space in a spine of a patient;
attaching the coupling of each support plate to a corresponding one of the disc beams in the vertebral interbody space.

7. A method as in claim 6 wherein the upper support plate is a pair of upper support plates and the lower support plate is a pair of lower support plates and the attaching of the support plates includes attaching each of pair of upper support plates to opposite beams on the upper disc beam and attaching each of a pair of lower support plates to opposite beams on the lower disc beam.

8. A method as in claim 6 wherein the interbody space is between two adjacent vertebra and the method further comprises positioning the upper support plate opposite to a lower surface of an upper vertebra and positioning the lower support plate opposite to an upper surface of a lower vertebra.

9. A method as in claim 6 wherein the disc beams and support plates are sequentially surgically inserted posteriorly into a patient.

10. An artificial spine disc assembly comprising:
a first disc beam having a center block with a rounded joint surface and a pair of opposite beams extending outward from the center block;
a second disc beam having a center block with a second joint surface and a pair of opposite beams extending outward from the center block, wherein the second joint surface seats into the first joint surface when the second disc beam is mounted on the first disc beam;
a first pair of spine support plates, wherein each plate has a generally planer surface adapted to face and engage a vertebra and a coupling to engage one of the opposite beams of the first disc beam, and
a second pair of spine support plates, wherein each plate has a generally planer surface adapted to face and engage a vertebra and a coupling to engage one of the opposite beams of the first disc beam.

11. An artificial spine disc assembly as in claim 10 wherein the first joint surface is a concave surface in an upper face of the center block of the second disc beam, and the second joint surface is a convex surface on a lower face of the center block of the first disc beam.

12. An artificial spine disc assembly as in claim 11 wherein the concave surface is a rounded well in the upper face and the convex surface is a semi-hemispherical surface in the lower face.

13. An artificial spine disc assembly as in claim 11 packaged with the disc beams and spine support plates disassembled in a sterile package to be shipped to a medical service provider.
